(19) United States
(12) Patent Application Publication
(10) Pub. No.: US 2025/0072925 A1
(43) Pub. Date: Mar. 6, 2025

(54) PERICARDIAL TRANSECTION DEVICES WITH PLURALITY OF INCISION MEMBERS AND METHODS OF REDUCING PERICARDIAL RESTRAINT

(71) Applicant: EDWARDS LIFESCIENCES CORPORATION, Irvine, CA (US)

(72) Inventors: Bezalel Haberman-Browns, Pardes Hanna (IL); Mazen Saadi Albaghdadi, Irvine, CA (US)

(21) Appl. No.: 18/947,947

(22) Filed: Nov. 14, 2024

Related U.S. Application Data
(63) Continuation of application No. PCT/US2023/023753, filed on May 26, 2023.
(60) Provisional application No. 63/347,527, filed on May 31, 2022.

(51) Int. Cl. A61B 17/32; A61B 17/00; A61B 90/00

(52) U.S. Cl. CPC A61B 17/320016; A61B 2017/00243; A61B 2017/00336; A61B 2017/00867; A61B 2090/3966

(57) ABSTRACT

Several exemplary transection devices are disclosed, comprising an elongated member having a distal end and a proximal end along a longitudinal axis, the proximal end coupled to an end of a catheter, a plurality of extensions operably coupled to the distal end of the elongated member, a sheath having a distal end encircling the elongated member in a longitudinally slidable relation, where in a first sliding position, the elongated member and the plurality of extensions are encircled by the sheath; and a second sliding position, the plurality of extensions projects from the distal end of the sheath. Such devices are useful for deployment in the pericardial cavity and making incisions through the pericardial membrane or parietal layer of the pericardium. These examples share the characteristic that they are deployed intravascularly through the RA, RAA, IVC, SVC, CS, or via a subxiphoid approach.

### RELATED APPLICATIONS

This application is a continuation application of International Patent Application No. PCT/US2023/023753, filed May 26, 2023, which claims the benefit of U.S. Provisional Application No. 63/347,527, filed on May 31, 2022. Each of the foregoing patent applications are hereby incorporated by reference in their entireties.

### TECHNICAL FIELD

This disclosure is directed to methods for treating heart failure, for example, heart failure with preserved ejection fraction (HFpEF) or reduced ejection fraction (HFrEF) by providing pericardial transection devices that introduce one or more incision lengths in a pericardium, e.g., a pericardial layer, fibrous layer, and/or adipose tissue.

### BACKGROUND

Pericardial restraint is a normal physiologic process that becomes exaggerated, for example, in some patients with heart failure with preserved ejection fraction (HFpEF) and causes the right heart to run out of space when filling, thereby squeezing and over pressurizing the left heart during physical activity in these patients. The increased left heart pressure backs up into the lungs and causes these patients to experience significant breathing difficulties when trying to do minimal activity, (exertional dyspnea). Exertional dyspnea is the most common symptom in patients with HFpEF and the most common cause for admission to the hospital in patients with HF in general. Currently, there is no therapeutic option for patients with HFpEF that specifically targets pericardial restraint.

### SUMMARY

In a first example, a pericardial transection device is provided, the device comprising a plurality of extension members at least one of which defining a proximal end projecting distally from a catheter, each of the plurality of extension members defining a distal end operably coupled to a respective incision assembly, and a sheath surrounding each of the extension members. Where the sheath translates relative the plurality of extension members to at least a first sliding position at which each of the plurality of extension members are disposed within the sheath, and a second sliding position at which at least one of plurality of extension members projects outwardly from the distal end of the sheath.

In one aspect, the each of the plurality of extension members extend from the catheter. In another aspect, alone or in combination with any of the previous aspects, the catheter defines a plurality of lumens such that each of the plurality of extension members extend from respective lumens of the plurality of lumens.

In another aspect, alone or in combination with any of the previous aspects, the at least one of the extension members of the plurality of extension members is steerable. In another aspect, alone or in combination with any of the previous aspects, the each of the extension members of the plurality of extension members is steerable.

In another aspect, alone or in combination with any of the previous aspects, the device further comprises a controller operably coupled with at least one flexible member of the plurality of extension members, wherein the at least one flexible member is steerable in response to an instruction from the controller. In another aspect, alone or in combination with any of the previous aspects, the device further comprises a controller operably coupled with each of the plurality of extension members, wherein each of the plurality of extension members is independently steerable in response to respective instructions from the controller.

In another aspect, alone or in combination with any of the previous aspects, the at least one flexible member of the plurality of extension members is formed of a plurality of connected segments.

In another aspect, alone or in combination with any of the previous aspects, the at least one flexible member of the plurality of extension members defines one or more apertures providing fluid communication between an interior of the flexible member and an exterior of the pericardial transection device or a source of vacuum.

In another aspect, alone or in combination with any of the previous aspects, the sheath defines a circular cross-sectional shape such that a distal end of the sheath encircles each of the plurality of extension members. In another aspect, alone or in combination with any of the previous aspects, the sheath further encircles each of the respective incision assemblies.

In another aspect, alone or in combination with any of the previous aspects, the any of the sheath, the plurality of extension members, and the incision assemblies comprises a radiopaque material that is randomly dispersed or arranged in a pattern.

In another aspect, alone or in combination with any of the previous aspects, the at least one of the incision assemblies comprises one or more electrodes. In another aspect, alone or in combination with any of the previous aspects, the at least one of the incision assemblies comprises an edged cutting tool.

In another aspect, alone or in combination with any of the previous aspects, the at least two of the plurality of extension members are structured to present in a curvilinear arc relationship about a heart in the second position. In another aspect, alone or in combination with any of the previous aspects, the incision assemblies are staggered in a curvilinear arc relationship in a same plane.

In another aspect, alone or in combination with any of the previous aspects, the at least one of the plurality of extension members extends coaxial with the catheter and at least one of the plurality of extension members extends opposed to the catheter. In another aspect, alone or in combination with any of the previous aspects, the at least two of the plurality of extension members extend in different planes to provide fixation and/or increase surface contact with a heart.

In another aspect, alone or in combination with any of the previous aspects, the plurality of extension members present in a curvilinear arc arrangement in the second sliding configuration. In another aspect, alone or in combination with any of the previous aspects, the at least a pair of the plurality of extension members present in a curvilinear arc that projects outwardly in opposed directions in the second sliding configuration.

In another aspect, alone or in combination with any of the previous aspects, the plurality of extension members present in a curvilinear arrangement and the incision assemblies present in a fixed, spatially separated relationship to each other in the second sliding configuration. In another aspect, alone or in combination with any of the previous aspects, the which at least a pair of the plurality of extension members present in a curvilinear arc that projects outwardly in opposed directions and the incision assemblies present in a fixed, spatially separated relationship to each other in the second sliding configuration.

In another aspect, alone or in combination with any of the previous aspects, the device further comprises a controller, wherein each of the incision assemblies are independently operably coupled to the controller.

In another aspect, alone or in combination with any of the previous aspects, the device is sterilized.

In another example, a pericardial transection device is provided, the device comprising an elongated member having a distal end and a proximal end along a longitudinal axis, the proximal end coupled to an end of a catheter, one or more extensions operably coupled to the distal end of the elongated member, a sheath having a distal end encircling the elongated member in a longitudinally slidable relation, where in a first sliding position, the elongated member and the one or more extensions are encircled by the sheath; and a second sliding position, the one or more extensions projects from the distal end of the sheath.

In one aspect, the elongated member is a shape memory material. In another aspect, alone or in combination with any of the previous aspects, the elongated member is flexibly rigid.

In another aspect, alone or in combination with any of the previous aspects, the one or more extensions are a shape memory material. In another aspect, alone or in combination with any of the previous aspects, the one or more extensions are flexibly rigid.

In another aspect, alone or in combination with any of the previous aspects, the one or more extensions are spatially separated in a plane in the second sliding position. In another aspect, alone or in combination with any of the previous aspects, the one or more extensions extend in different planes in the second sliding position to provide fixation in the pericardial space and to provide simultaneous contact with the parietal layer.

In another aspect, alone or in combination with any of the previous aspects, the one or more extensions extend in a X-or Y-or T-shape in the second sliding position. In another aspect, alone or in combination with any of the previous aspects, the X-or Y-or T-shape comprises at least one of the one or more extensions having an incision member and at least two of the one or more extensions absent an incision member to provide any one of stability, fixation or increase parietal layer contact in the second sliding position.

In another aspect, alone or in combination with any of the previous aspects, the at least two of the one or more extensions extend obtuse to each other in the second sliding position. In another aspect, alone or in combination with any of the previous aspects, the at least two of the one or more extensions extend acute to each other in the second sliding position. In another aspect, alone or in combination with any of the previous aspects, the at least two of the one or more extensions extend orthogonal to each other in the second sliding position.

In another aspect, alone or in combination with any of the previous aspects, the at least one of the one or more extensions comprises an incision member. In another aspect, alone or in combination with any of the previous aspects, the sheath comprises a radiopaque material that is randomly dispersed or arranged in a pattern.

In another aspect, alone or in combination with any of the previous aspects, the at least one of the one or more extensions comprises one or more electrodes. In another aspect, alone or in combination with any of the previous aspects, the at least one of the incision assemblies comprises an edged cutting tool. In another aspect, alone or in combination with any of the previous aspects, the incision member comprises a blade, an electrode, a RF electrode, or combination, capable of cutting pericardial tissue.

In another aspect, alone or in combination with any of the previous aspects, the device is sterilized.

In yet another example, a method of incising pericardial tissue in a subject in need thereof is provided, the method comprising providing a pericardial device of any one of the previous examples, introducing the pericardial device to a pericardial cavity, and incising at least a portion of a parietal layer of a pericardium along a length and a path.

In one aspect, the method further comprises reducing pericardial restraint of the subject. In another aspect, alone or in combination with any of the previous aspects, the pericardial device is introduced subxiphoidally. In another aspect, alone or in combination with any of the previous aspects, the pericardial device is introduced transvascularly.

In another aspect, alone or in combination with any of the previous aspects, the pericardial device is introduced transvascularly via the Superior Vena Cava. In another aspect, alone or in combination with any of the previous aspects, the pericardial device is introduced transvascularly via the Inferior Vena Cava.

In another aspect, alone or in combination with any of the previous aspects, the incising of at least a portion of the parietal layer is by reverse cutting along a path and a length. In another aspect, alone or in combination with any of the previous aspects, the method further comprises repeating the step of incising the pericardial cavity along a different length, a different path, or a different length and a different path.

In another aspect, alone or in combination with any of the previous aspects, the method further comprises, after the introducing step and before the incising step, puncturing out of the pericardial cavity and exposing the incision portion.

### BRIEF DESCRIPTION OF THE DRAWINGS

In order to understand and to see how the present disclosure may be carried out in practice, examples will now be described, by way of non-limiting examples only, with reference to the accompanying drawings, in which:

FIG. 1A is a sectional view of a 4-chambered heart.

FIG. 1B is an enlarged view of section 1B of FIG. 1A depicting the layers of the heart wall, including the pericardial cavity.

FIG. 1C is a further enlarged view of section 1C of FIG. 1A depicting the serosal, visceral, fibrous layers and adipose tissue of the parietal pericardium, including the pericardial cavity.

FIGS. 2A-2B depicts exemplary pericardial layer tissue transection device in first configuration and alterative second configuration, as disclosed or described herein.

FIG. 2C is a section view of FIG. 2B along section line 2C-2C.

FIG. 2D is a section view of FIG. 2B along section line 2C-2C in an alternative structural configuration.

FIGS. 3A-3B depict another example of a pericardial layer tissue transection device in first configuration and second configuration, as disclosed and described herein.

FIG. 3C is a section view of FIG. 3B along section line 3C-3C.

FIG. 3D is a section view of FIG. 3B along section line 3D-3D.

FIG. 4 is a simplified diagram of a parietal layer incision length and cut path from an exemplary pericardial layer tissue transection device as disclosed and described herein.

FIGS. 5A-5C depict another example of a pericardial layer tissue transection device in first configuration and alterative second configuration, as disclosed and described herein.

FIG. 6 is a simplified diagram of a parietal layer incision length and cut path from an exemplary pericardial layer tissue transection device as disclosed and described herein.

FIGS. 7A, 7B and 7C depict a visualization system for use in a multi-lumen catheter device in combination with the presently disclosed pericardial layer tissue transection devices.

FIGS. 8A and 8B depict exemplary controller devices for delivering the incision devices disclosed and described herein.

FIG. 9 is a simplified diagram of a multi-lumen catheter based pericardial layer tissue transection device approach to the pericardial cavity, as disclosed and described herein.

FIG. 10 is a simplified diagram of an alternative multi-lumen catheter based pericardial layer tissue transection device approach to the pericardial cavity, as disclosed and described herein.

### DETAILED DESCRIPTION

Several exemplary devices for making incisions through the pericardial membrane or parietal layer of the pericardium are described. These examples share the characteristic that they are deployed intravascularly through the Inferior Vena Cava (IVC), Superior Vena Cava (SVC), Coronary Sinus (CS), right atrium (RA), right atrium appendage (RAA), or via a subxiphoid approach.

As used herein the phrase “pericardial space” and pericardial cavity are used interchangeably and are inclusive of their ordinary and customary meaning to one of ordinary skill in medical and surgical arts, for example, a space, cavity, or liquid medium generally disposed between the parietal pericardium and visceral pericardium of a mammalian heart.

As used herein the phrase “pericardial tissue” is inclusive of its ordinary and customary meaning to one of ordinary skill in medical and surgical arts, for example, tissue associated with the pericardium.

As used herein, unless otherwise specified, the phrase “parietal layer” comprises at least the serosal and fibrous layer of the parietal pericardium, and optionally adipose tissue contained between, below, above, or within said layers. Further, the phrase “parietal layer” is inclusive of the ordinary and customary meaning to one of ordinary skill in medical and surgical arts, for example tissue layers generally disposed the adjacent to and including adipose tissue within and outside the pericardial cavity and superficial to the visceral layer of the pericardium.

As used herein the phrase “cutting surface” is inclusive of one or more of an edge of a sharpened blade or the surface of an electrode structured to receive sufficient current or radio frequency energy (RF) to ablate, burn, vaporize, or separate tissue. A cutting surface can be inclusive of both a sharpened edge and an electrode.

As used herein the phrase “reverse cutting” and “pull-back cutting” are used interchangeably and refer to methods involving the presentation of a cutting surface to tissue, the cutting surface adjacent a distal end of a multi-lumen catheter device or catheter, and the application of a directional force sufficient to cut or separate the tissue, the force being substantially in a direction towards the proximal end of the multi-lumen catheter device or catheter, for example, by pulling the multi-lumen catheter device or catheter while the cutting surface is engaged with the tissue.

It should be understood that the term “cutting” used herein refers to tissue disruption, for example, a sharp-cutting incision of the type associated with a knife blade such as a scalpel blade, or an electrosurgical device that provides electrical current to an electrically conductive material or electrode sufficient to disrupt tissue. The term “cutting” used herein includes “filet”, “slicing”, and the like.

As used herein the phrase “incision length” is inclusive of a non-zero distance of a cut or incision, for example, beginning at a first point, e.g., a target point, and terminating at a second point, e.g., an end point. An incision length can be linear, non-linear, or a plurality of linear and/or non-linear lengths that intersect or do not intersect about a curved or non-planar surface, such a heart.

As used herein the phrase “reducing pressure” and “reducing restraint” are inclusive of their ordinary and customary meaning of one to ordinary skill in medical and surgical arts.

As used herein the phrase “preserved ejection fraction” is inclusive of the ordinary and customary meaning to one of ordinary skill in medical and surgical arts, for example, a clinical syndrome in which patients display signs and symptoms of heart failure as the result of high left ventricular (LV) filling pressure despite normal or near normal left ventricle (LV) ejection fraction (LVEF; ≥50 percent).

As used herein the phrase “heart dysfunction” is inclusive of the ordinary and customary meaning to one of ordinary skill in medical and surgical arts, for example, heart failure or congestive heart failure.

As used herein the phrase “pericardial transection device” is inclusive of a device with an incision surface, for example an edge of a blade or a surface of an energized electrode.

As used herein the phrase “pericardial incision assembly” and “incision assembly” are used interchangeable and refer to an assemblage that includes a pericardial transection device.

As used herein the phrase “multi-lumen catheter device” is inclusive of a catheter structured with at least one lumen comprising a medical instrument, device, or component thereof, for example, a pericardial transection device.

As used herein, the terms “first,” “second,” and the like are only used to describe elements as they relate to one another, and are in no way meant to recite specific orientations of an article or apparatus, to indicate or imply necessary or required orientations of an article or apparatus, to indicate or imply necessary or required configurations of an article or apparatus, or to specify how an article or apparatus described herein will be used, deployed, transitioned from different configurations, or positioned in use.

As used herein, when an element is referred to as being “adjacent” and “coupled” when referring to two structures or layers, the two structures or layers are in proximity with one another with no intervening open space between them.

As used herein, when an element is referred to as being “coupled” or “adjacent” to another element, the two elements or structures are in proximity with one another, however, other elements or intervening elements may be present.

As used herein, when an element is referred to as being “directly coupled” or “directly adjacent” to another element, other elements or intervening elements are not present.

As used herein, term “operably coupled”, includes direct coupling and indirect coupling via another component, element, circuit, or structure and/or indirect coupling between items via an intervening item.

As used herein the phrase “nerve stimulation device” is inclusive of a device capable of applying an electrical potential to a nerve and to cause an observable effect that is directly or indirectly correlated to the applied potential, for example a pacing probe stimulating a phrenic nerve and causing an observable breathing disruption.

As used herein the phrase “nerve detecting device” is inclusive of a device capable of establishing a location or locale of at least part of a nerve and providing location or proximity information with no or substantially no physical effect or stimulus on the nerve, for example, an impedance sensor for detecting an electrical field generated by a nerve and to correlate, directly or indirectly, the location or proximity of the nerve relative to the impedance sensor.

As used herein the term “actuator” is inclusive of a mechanism for triggering an action.

As used herein the term “controller” is inclusive of a device having an actuator.

As used herein the phrase “biasing member” is inclusive of a device configurable in a stored energy state and a released energy state, for example, a spring.

As used herein the phrase “stabilizing member” is inclusive of a device configurable to impart stability and/or securement of a device to or within a structure, such as for example, stabilizing or securing a cutting surface positioned in a pericardial cavity from rolling, twisting, buckling and/or oscillating prior to or during use.

As used herein, “delta pulmonary capillary wedge pressure, Δ(PCWP)” is the difference between pulmonary capillary wedge pressure (PCWP) measured on volume challenge (i.e. leg raise and/or fluid administration) and PCWP at rest.

As used herein the phrase “puncturing tip” is inclusive of an atraumatic object suitable for puncturing or penetrating tissue without substantial trauma to or bleeding from the vicinity of the picture or penetration.

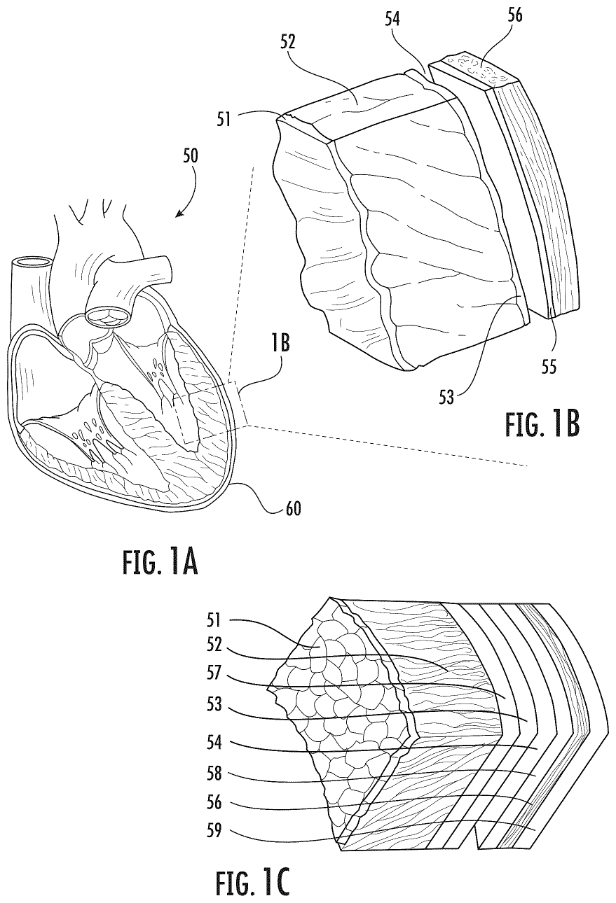

With reference to FIGS. 1A, 1B, 1C, and section 1C, layers of a heart wall of a heart 50, from inside-out, being the endocardium 51, the myocardium 52, epicardial adipose tissue 57, the visceral layer 53 of the serous pericardium 58, the pericardial cavity 54, the parietal layer 55 of the serous pericardium 58, and the fibrous pericardium 56, and pericardial adipose tissue 59 are depicted. In one example, the presently disclosed devices are structured for introduction to the pericardial cavity 54 and for cutting tissue layers generally disposed adjacent to and including adipose tissue within and outside the pericardial cavity and superficial to the visceral layer 53 of the pericardium.

The presently disclosed pericardial tissue transection devices includes a perforating or puncturing portion designed to initially puncture the pericardial membrane. A guidewire, needle, microneedle knife, or electrical current may be used to form the perforation or puncturing of the pericardial membrane to allow access of the pericardial transection device to the pericardial cavity. Once the pericardial membrane is punctured, an incision assembly adjacent a distal end of a catheter or multi-lumen catheter is manipulated to a location within the pericardial cavity, and an incision member is allowed to engage with the pericardial tissue and an incision is created upon retraction of the pericardial tissue transection device towards the point of entry into the pericardial membrane. The incision member may alternatively, or in combination with a sharp edge, utilize RF energy to facilitate ease of incising and for providing some hemostasis or coagulation of incised edges of the pericardial membrane.

Several pericardial tissue transection device examples are shown in the attached figures. Hereinafter, the phrase “pericardial tissue transection device” and “transection device” shall be used interchangeably. Each transection device would be first introduced into the pericardial space via a transvascular or subxiphoid approach.

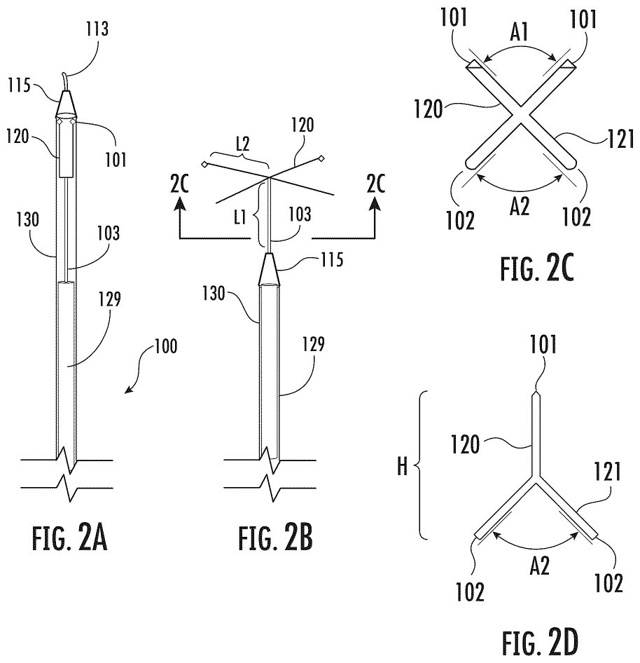

With reference to FIGS. 2A-2D, an exemplary pericardial tissue transection device 100 is shown. Device 100 has a “rake” or “sled” style configuration. Device 100 includes an atraumatic tip 115 designed to follow an initial puncture of the pericardial membrane. Atraumatic tip 115 can be structured to receive a guidewire 113 to facilitate introduction of the device to the pericardial membrane after initial puncture by a needle etc. Once the membrane is accessed, device 100 is maneuvered in the pericardial cavity 54, sheath 130 is retracted to release extensions 120 coupled to member 103, extensions 129 having the plurality of spatially separated or staggered incision members 101 to a starting point of an incision path, and the incision member 103 engages the membrane and, upon retraction towards the point of entry into the pericardial space, creates an incision in the parietal layer in at least the parietal layer of a length and along a path. In one example, the length and path of the incision is determined preoperatively.

The incision member 103 may be a sharpened edge, utilize RF energy or current, or a combination of sharpened edge/RF/current to facilitate ease of cut and hemostasis of the pericardial membrane. In one example, member 103 and coupled extensions 120 are shape-memory material that can be structured in a collapsed state for delivery, deployed by sheath retraction, and then re-collapsed by the sheath 130.

Device 100 comprises an elongated body 129 with proximal end and a distal end, a longitudinal axis, a circumference, and extensions 120 coupled to member 103 within the elongated body 129 and generally parallel with the longitudinal axis. Atraumatic tip 115 tapers to provide assistance with entry to the pericardial tissue or through vascular or heart tissue. In one example, the elongated body 129 is tubular. In another example, the elongated body 129 is a flexible multi-lumen catheter with at least two lumens. The flexible catheter can be a steerable catheter or a steerable multi-lumen catheter with at least two lumens.

In one example, the elongated body 129 comprises at least on lumen receiving a guidewire 113. In one example, the elongated body 129 has an outer diameter between about 6 Fr to about 30 Fr (about 2 mm to about 10 mm). In some examples, the elongated body 129 has an outer diameter between about 7 to about 12 Fr. In some examples, the elongated body 129 with sheath 130 has an outer diameter less than about 10 Fr.

Member 103 has a distal end and a proximal end integral with the elongated body 129 of a length L1 (1-5 mm, for example). As shown, distal end of member 103 is coupled to extensions 120. At least a portion of the extension can comprise radiopaque material randomly dispersed or arranged in a pattern for visualization using conventional visualization techniques during use.

The incision member 101 of transection device 100 extends distal ends of extensions 120. In one example, a plurality of incision member 101 are positioned along the length L2 of extensions 120 from at least a portion of the wall surface. As shown, the incision member 103 extends from the elongated body 129 to the extension at an obtuse angle relative to the longitudinal axis. In another example, the incision member 103 can extend from the elongated body 129 to the extension 120 at an acute angle or at a right angle.

As shown in FIG. 2C, in section view of FIG. 2B alone section line 2C-2C, transection device 100 can be structured to utilize extensions as stabilizing members and for transection of at least the pericardial layer. Thus, in one example, extensions 120, 121 depicting an “X” configuration are shown, where incision members 101 are positioned on adjacent extensions 120, so that during use, they face the underside of the parietal layer 55 while remaining extensions 121 and elements 102 provide support, low friction, and/or stabilization, e.g. preventing rolling or yaw. In one example, elements 102 are sensors and can indicate to a user the orientation of the incision members 103, for example, using echocardiograph, impedance, or other signaling. In one example, extensions 120 are independently flexibly rigid. In one example, extensions 120 project angularly at an angle A1 and extensions project angularly at an angle A2. In one example, angles A1, A2 are independently obtuse, acute, or orthogonal. In another example, A1 is larger, the same, or smaller than angle A2. In one example, extensions 120 comprising incision members 103 are more rigid or stiffer or thicker than extensions 121. In one example, angle A2 is more variable than angle A1 due to a difference in flexibility, stiffness, or thickness of extensions 121.

As shown in FIG. 2C, in section view of FIG. 2B alone section line 2D-2D, transection device 100 extensions 120, 121 are depicted in an “Y” configuration, where incision member 101 is positioned on extensions 120, so that during use, it faces the underside of the parietal layer 55 while remaining extensions 121 and elements 102 provide support, low friction, and/or stabilization, e.g. preventing rolling or yaw. In one example, elements 102 are sensors and can indicate to a user the orientation of the incision members 103, for example, using echocardiograph, impedance, or other signaling. In one example, extensions 120 are independently flexibly rigid. With reference to FIG. 2D, extensions 121 project angularly at an angle A2 that can be obtuse, acute, or orthogonal. In one example, the height H of the rake of device 100 is, for example, 1-7 mm, 2 to 6 mm, or 3 to 5 mm (referring to the vertical distance between incision member 101 and elements 102 as it will be presented to the pericardial cavity 54 in the second sliding position). In one example, at least a portion of the incision member 103 is a sharpened edge or is energizable with electrical current or radio frequency energy sufficient to separate pericardial tissue. In another example, the incision member 103 is a sharpened edge with at least a portion thereof energizable with electrical current or radio frequency energy sufficient to separate pericardial tissue. Other configurations, such as a “T” configuration can be used.

Transection device 100 is shown comprising a retractable sheath 130 structured to reversibly cover the extensions 120, 121 and the incision member(s) 101 during manipulation through the pericardial cavity 54. Retractable sheath 130 is structured to be withdrawn to expose the extension members 120, 121 and incision members 101 so as to receive and to cut pericardial tissue. Thus, in one example, the sheath is structured to transition between a first sliding position at which each of the extensions are disposed within the sheath, and a second sliding position at which the extensions project outwardly from the distal end of the sheath 130. When desired, retractable sheath 130 can be advanced forward to collapse and cover the extension members 120, 121 for removal of the device. In one example, sheath 130 defines a circular cross-sectional shape such that a distal end of the sheath encircles each extension members 120.

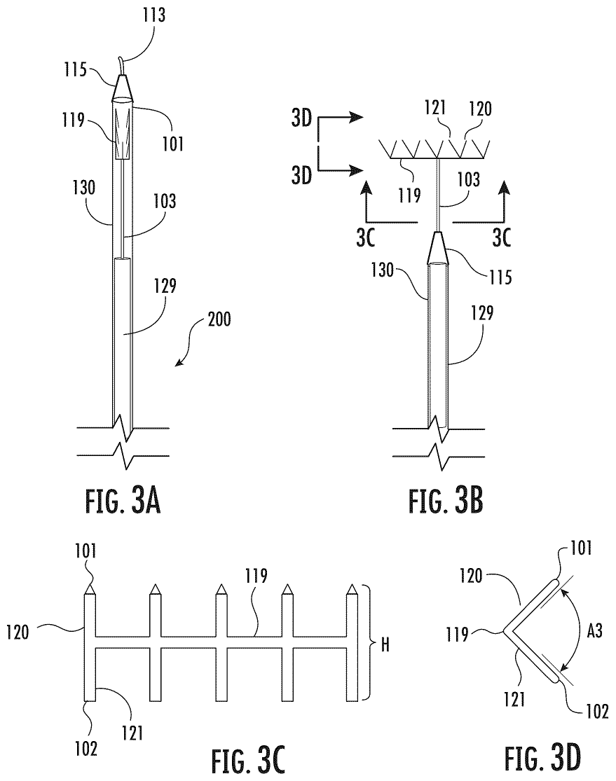

In an alternative example, and with reference to FIGS. 3A-3D, transection device 200 is depicted having similar structural elements as device 100 but with a “comb” like structural arrangement of the member 103 and extensions 120, 121 being coupled by member 119. Member 119 is shown essentially orthogonal to member 103, however, angular deviations from orthogonal are envisioned. In one example, member 103, member one 19, and extensions 120, 121 are of a shape memory material so as to provide for collapsing in a catheter or sheath for delivery. In a similar manner to that of device 100 described above, device 200 is shown comprising a retractable sheath 130 structured to reversibly cover the member 103, and extension members 119, 120, 121 and the incision member(s) 101 during manipulation through the pericardial cavity 54. Retractable sheath 130 is structured to be withdrawn to expose at least the extension members 119, 120, 121 and incision members 101 so as to receive and to cut pericardial tissue. Thus, in one example, the sheath is structured to transition between a first sliding position at which each of the extension members are disposed within the sheath, and a second sliding position at which the extension members project outwardly from the distal end of the sheath 130. When desired, retractable sheath 130 can be advanced forward to collapse and cover the extension members 119, 120, 121 and incision members 101 for removal of the device.

As shown in FIGS. 3C, 3D, extension members 119, 120, and 121 provide a comb like arrangement of incision members 101. In one example, the height H of the comb is, for example, 1-7 mm, 2 to 6 mm, or 3 to 5 mm (referring to the vertical distance between incision member 101 and element 102 as it will be presented to the pericardial cavity 54 in the second sliding position). Extension members 120, 121 can be angularly disposed relative to one another about extensions member 119 by angle A3, as depicted in FIG. 3D. Angle A3 can be between 45° to 180°.

With regard to devices 100, 200, at least a portion of the sheath 130 can comprise radiopaque material randomly distributed or arranged in a pattern for visualization using conventional visualization techniques during use. Transection device 100 is shown further comprising an atraumatic tip 115 adjacent the distal end of the elongated body 129. Atraumatic tip 115 is structured to receive a guidewire 113 and taper to be flush with the guidewire. At least a portion of the atraumatic tip can comprise radiopaque material randomly distributed or arranged in a pattern for visualization using conventional visualization techniques during use.

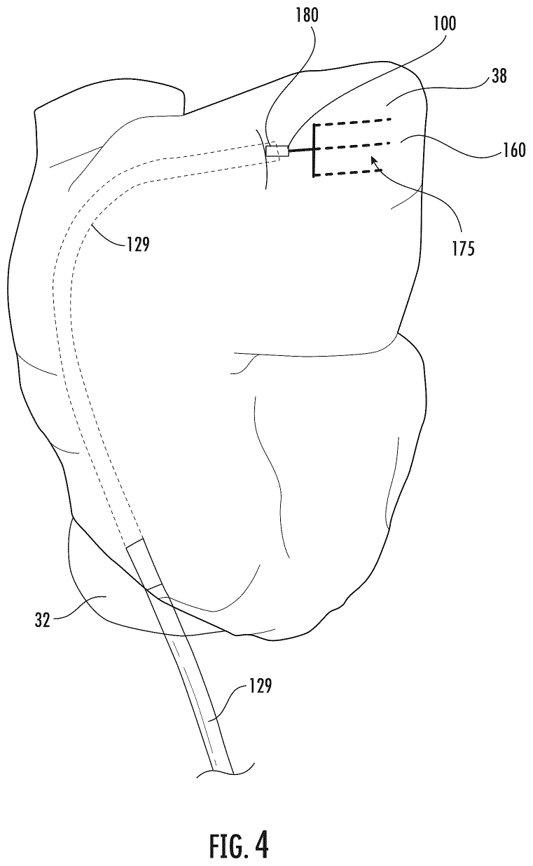

With reference to FIG. 4, use of device 100, 200 is shown in a transvascular entry through entry point 180 of the right atrial appendage 38 to the pericardial cavity 54 to a starting point 160 and the creation of parallel incisions 175 in the parietal layer back to the entry point 180. A subxiphoid approach can be used to present device 100, 200. Other cut paths and lengths can be used.

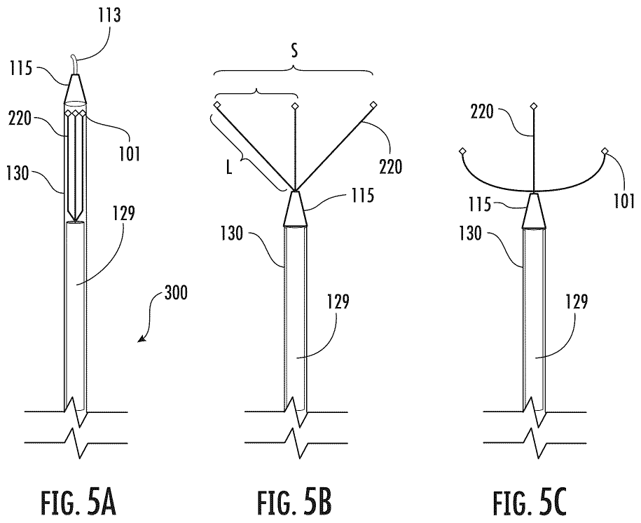

With reference to FIGS. 5A-5C, another exemplary transection device 300 is shown, where device 300 comprises multiple, independently steerable catheters that can be positioned in various orientations over the heart or within the pericardial cavity to enable cutting the pericardium in various trajectories without having to re-position the cutting catheter.

Transection device 300 comprise a retractable sheath 130 covering the plurality of extension members 220. Retractable sheath 130 is structured to be withdrawn to expose the extension members 220 a length L (e.g., 1-10 mm or more) and spatially separate incision members 101 by a variable distance S so as to present to and cut pericardial tissue. Thus, in one example, the sheath 130 is structured to transition between a first sliding position at which each of the extensions are disposed within the sheath, and a second sliding position at which the extensions project outwardly from the distal end of the sheath 130. When desired, retractable sheath 130 can be advanced forward to collapse and cover the extension members 120 and incision members 101 for removal of the device.

In one example, at least one of the extension members 120 of device 300 is steerable. The extension member is steerable in response to an instruction from a controller, for example, and each extension member can be independently steerable in response to respective instructions from the controller. In one example, one or more of the extension members 120 are formed of a plurality of connected segments.

In one example, one or more side ports or apertures are provided to catheter 129 for suction for stabilizing the device, and/or to introduce fluid e.g., contrast fluid, for visual guidance.

In one example, extension members 120 comprise conductive wire with a plurality of spaced apart skive portions through a dielectric coating. In yet another example, the conductive wire comprises a continuous elongated skive portion through the dielectric coating. At least a portion of the dielectrically coated conductive wire can comprise radiopaque material randomly distributed or arranged in a pattern for visualization using conventional visualization techniques during use.

Orientation of the incision members can be achieved using imaging in combination with radiopaque marking of selected wires, for example. This method can be further adapted to provide a specific geometry or pattern of cut as desired to achieve optimal reductions in the intracardiac pressures.

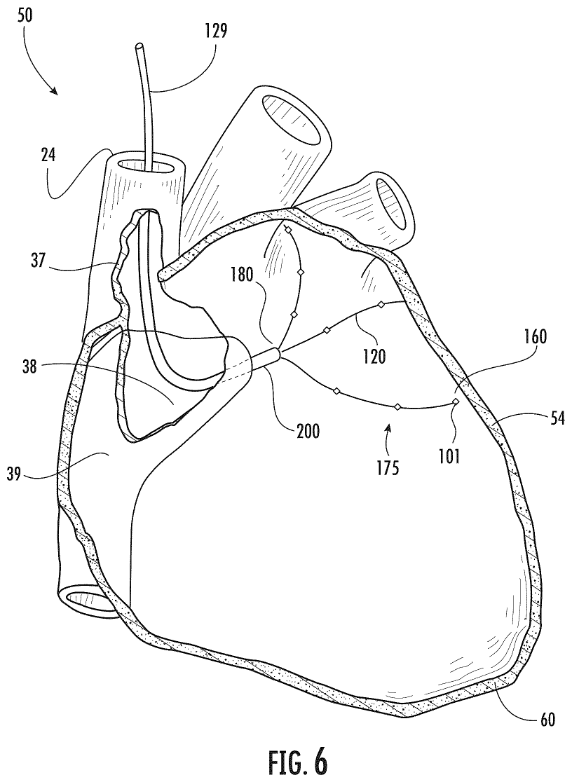

With reference to FIG. 6, use of device 300 is shown in a transvascular entry via the SVC 24 and entry point 180 through right atrial appendage 38 to the pericardial cavity 54 and the placement of extension members 120 in a curvilinear arc arrangement. In this configuration, a plurality of incisions cavity can be created simultaneously or sequentially without substantial movement of the extension members 120. Alternatively, extension members 120 can be simultaneously or sequentially pulled back towards entry point 180 from starting point 160 to provide multiple incisions 175 in the parietal layer along a path, which may be preoperatively determined.

In any of the examples described above, a puncture to deliver a guidewire into the pericardial space is performed through heart tissue. When a transvascular approach through the RAA, IVC, SVC or CS is employed, a closure device may be subsequently introduced for hemostasis at the conclusion of the procedure. In one example, the closure device includes outward or radially directed splines deployed in an expanded configuration. When the guide catheter is removed, the splines or radial members of the closure device contract inwardly towards the unstressed state of the transection device in order to close, occlude, and/or seal the opening. The closure device is designed such that a pericardial cutting device can pass through and into the pericardial space.

The following exemplary occlusion descriptions relate to a transvascular approach through the RAA, IVC, SVC or CS using one of the aforementioned transection devices 100, 300. In one example, an atraumatic tip 115 delivers a wire into the pericardial space through heart tissue. A closure or occlusion device is introduced for hemostasis during the procedure. The closure or occlusion device in one example includes outward or radially directed splines deployed in an expanded configuration. When the guide catheter is removed, the splines or radial members of the closure device contract inwardly towards the unstressed state of the transection device in order to close and seal the opening. The closure device is designed such that a pericardial cutting device can pass through and into the pericardial space.

With reference to FIGS. 1A, 1B, 1C, and sections 1B, 1C, layers of a heart wall of a heart 50, with pericardium 60, from inside-out, being the endocardium 51, the myocardium 52, epicardial adipose tissue 57, the visceral layer of the serous pericardium 58, the pericardial cavity 54, the parietal layer of the serous pericardium 58, and the fibrous pericardium 56, and pericardial adipose tissue 59 are depicted. In one example, the presently disclosed devices are structured for introduction to the pericardial cavity 54 and for cutting tissue layers generally disposed adjacent to and including adipose tissue within and outside the pericardial cavity 54 and superficial to the visceral layer 53 of the pericardium 60.

In one example, to provide orientational stability of the cutting surface to that of the parietal layer, an OTW introduction is employed for any of the previously disclosed devices, for example, whether through a dedicated lumen in multi-lumen catheter cross-section or ‘Rapid Exchange’ style catheter, or off-center attached cannula, or deflect-resistant catheter, as the delivering catheter randomly distributed or arranged in a pattern for visualization using conventional visualization techniques during use.

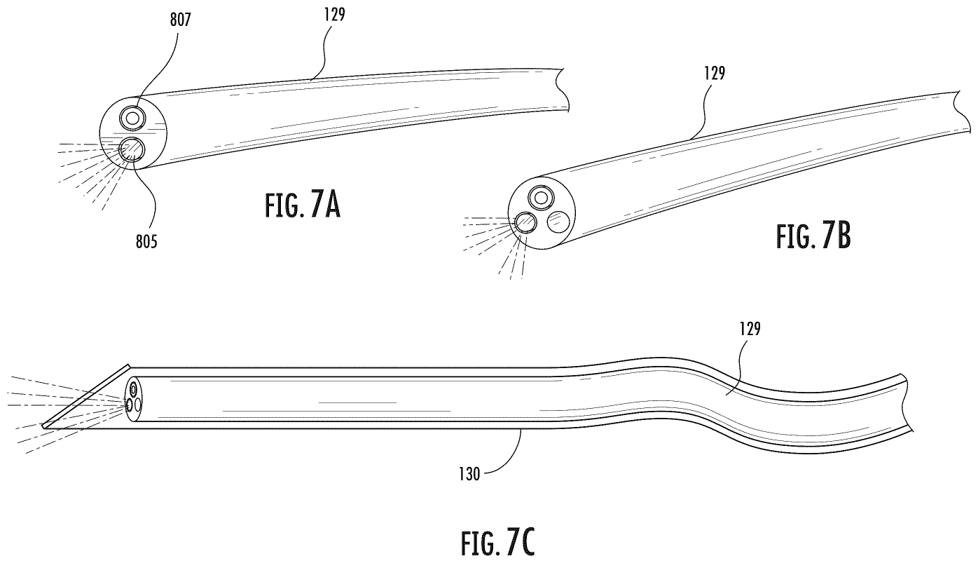

Current ECHO/fluoroscopy may not provide the required visualization for certain access applications of the presently disclosed transection devices, for example, gaining guidewire access pericardial cavity consistently and repeatedly may be desired. Thus, in one example, the multi-lumen catheter device 129 coupled to the presently disclosed transection devices comprises direct visualization, as shown in FIGS. 7A-7C allowing the user to watch real-time the advancement of the transection device 100, 200, 300 through various tissue layers until the desired location is reached. As shown in FIGS. 7A-7C, multi-lumen catheter 129, shown without a transection device for clarity, comprises a fiber optic channel and lens 807 adjacent a fiber optic channel 805 to provide light and to provide an analog or digital image in a multi-lumen catheter 129.

Changes in tissue layers that may not be visible under ECHO/fluoroscopy may be easily distinguishable under direct visualization such as tissue (vessel access), myocardium/pericardium (pericardial cavity access), myocardium/pericardium (outside pericardium), among other anatomical features. In one example, the presently disclosed devices discussed above further comprise an optical channel in the multi-lumen catheter to accommodate a lens coupled to a fiber optic cable, optionally with a light source, e.g., an LED. In one example, the presently disclosed method further comprises obtaining visual information during accessing, traversal of the pericardial cavity, exiting and/or cutting, for example, using an optical channel in the multi-lumen catheter to accommodate a lens coupled to a fiber optic cable, optionally with a light source, e.g., an LED.

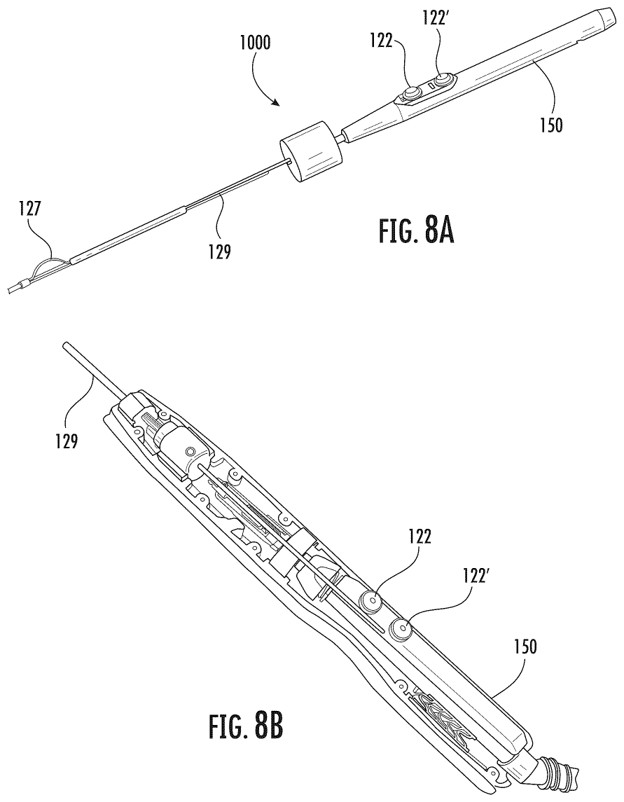

With reference to FIGS. 8A and 8B, a controller 1000 is shown having handle 150, actuating buttons 122, 122′ for operably coupling with the incision device, for example activating one or more electrodes, extending a wire, extending stabilizing members via a biasing member, etc. In one example, the controller 1000 allows the operation of various potential operations of the incision device 300, 400, 450, and 500, including extending and/or retracting a wire, which can be achieved by an appropriate mechanism structured to pull/push a rod. In one example, there is a mechanism used to release/retrieve balloons/nitinol components that function to stabilize and apply counterpressure for the incision device 300, 400, 450, and 500 and its components. Controller 1000 may include one or buttons used to operate and control the electrosurgical features of the device.

Although FIG. 8A depicts wire 127 as a portion of an incision device, one or more of the medical devices and/or incision devices shown and described herein with respect to FIGS. 2A-2C, 3A-3B, 5A-5C, may be coupled to and/or compatible with the controller 1000. Furthermore, although FIGS. 8A and 8B depict a particular controller 1000, the medical devices and/or incision devices shown and described herein with respect to FIGS. 2A-2C, 3A-3B, 5A-5C, may be coupled to and/or compatible with other controllers (e.g., from different manufacturers, vendors, distributors, and/or the like).

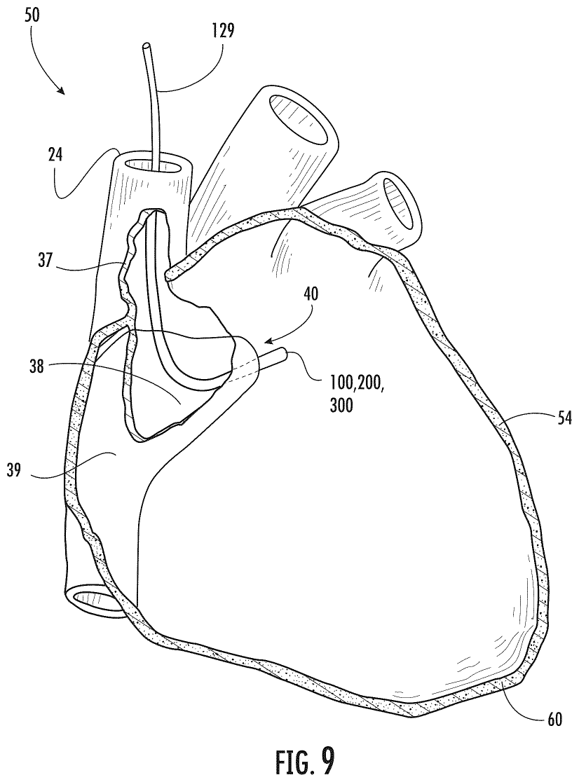

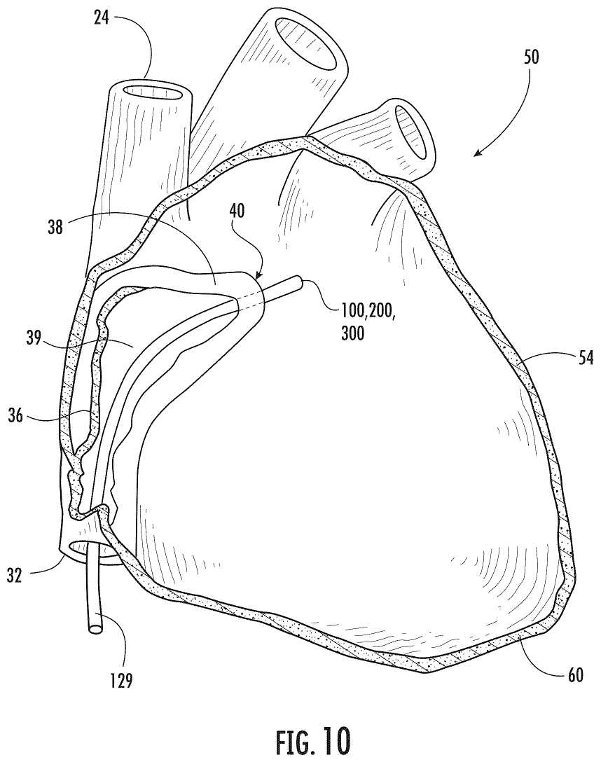

FIGS. 9 and 10 shows various intravascular approaches for delivering the transection devices of the present disclosure to the pericardial cavity 54. Thus, FIG. 9 depicts heart 50 viewed in isolation from the body, with the pericardium 60 or pericardial sac encasing the cardiac muscle (i.e., epicardium, myocardium, and endocardium). The small space which is present between the heart muscle and pericardium 60 represents the pericardial cavity 54.

The presently disclosed transection devices that can be presented to the pericardial cavity 54. In one example via the right atrial appendage 38 (RAA), which is a suitable site for entry into the pericardial cavity 54, is used. Right atrial appendage 38 lies tangential to and between pericardium 60 and the epicardium/epicardial adipose tissue 57. In one example, any of the presently disclosed devices is guided into right atrial appendage 38 via right atrium 39 so as to be positioned substantially in parallel with the wall of pericardium 60 such that when the wall of right atrial appendage 38 is pierced by the transection device 100, 200, 300 it is done substantially without risk of damaging the epicardium or other heart tissue.

In some examples, right atrial appendage 38 may be accessed via conventional vena cava routes. FIG. 9 illustrates entry of any of the presently disclosed devices into right atrium 39 via the superior vena cava 24 (SVC). A cut-away 37 shows passage of any of the presently disclosed devices through superior vena cava 24, right atrium 39, and right atrial appendage 38. A distal tip of catheter 129 is shown exiting right atrium 39 at apex 40.

FIG. 10 illustrates an alternative entry of any of the previously disclosed devices into right atrium 39 via the inferior vena cava 32 (IVC). A cut-away 36 shows passage of catheter 129 through inferior vena cava 32, right atrium 39, and right atrial appendage 38. A distal tip of catheter 129 is shown exiting right atrium 39 at apex 40.

Thus, by way of example, the method of the present disclosure includes the following steps. Any of the presently disclosed devices is maneuvered through one of the vena cava 24, 32 to right atrium 39. Once inside right atrium 39, any of the presently disclosed devices is passed into the right atrial appendage 38. The wall of right atrial appendage 38 is pierced at apex 40, and the catheter is advanced into the pericardial cavity 54. Other access routes to the pericardial cavity can be used, for example, direct “puncture out” of SVC or IVC/coronary sinus (CS) and a “puncture into” the pericardium.

Note that the wall of the right atrial appendage may be pierced with any of the presently disclosed devices itself, or with an instrument (e.g., guidewire) passed through a lumen of the any of the presently disclosed devices, e.g., over the wire. Further, any of the previously disclosed devices may be passed into the pericardial space through the opening in the wall of the atrial appendage, or an instrument passed through the lumen of any of the presently disclosed devices may be presented into the pericardial cavity 54. These details will depend on the procedure being performed and on the type of the previously disclosed device being employed.

In one example, device 100, 200, 300 is signal guided intravascularly and/or through the pericardium, pericardial membrane, and pericardial space, for example, using an injected contrast fluid (periodically or continuously) or radiopaque tracing, or using electrical signaling/detection means such as impedance or echocardiography.

In one example, the device 100, 200, 300 is structured to deliver a fluid or gas to the pericardial cavity 54 through a lumen of the catheter 129 so as to assist in the separation of some or all of the layers of the pericardial membrane or to expand the pericardial cavity 54, which may reduce pericardial constraint independently or in combination with transection.

In one example, the presently disclosed devices further comprise at least one nerve detection device. In one example, the at least one nerve detection device is located on the flexible catheter 129. In one example, the at least one nerve detection device is located adjacent the incision assembly. In one example, the at least one nerve detection device is located on the atraumatic tip 115 or sheath 130 distal end. In one example, the at least one nerve detection device is located on the incision member 101.

Any one of the presently disclosed devices further comprise at least one nerve stimulation device. In one example, the at least one nerve stimulation device is located on the flexible catheter 129. In one example, the at least one nerve stimulation device is located adjacent the incision assembly. In one example, the at least one nerve stimulation device is located on atraumatic tip 115 or sheath 130 distal end. In one example, the at least one nerve stimulation device is located adjacent the incision member 101.

Transection devices 100, 200, and 300 are structured for sterilization using conventional techniques such as ethylene oxide, electron beam, gamma, and autoclaving as well as chemical sterilization and aseptic manufacturing/packaging techniques.

A kit, comprising any one of the presently disclosed medical devices, a sheath 130, a guidewire 113, and an atraumatic tip 115 is provided.

While certain examples of the present disclosure have been illustrated with reference to specific combinations of elements, various other combinations may also be provided without departing from the teachings of the present disclosure. Thus, the present disclosure should not be construed as being limited to the particular exemplary examples described herein and illustrated in the Figures but may also encompass combinations of elements of the various illustrated examples and aspects thereof.

We claim:

1. A pericardial transection device for use in a pericardial cavity of a heart, said device comprising:
an elongated member having a distal end and a proximal end and extending along a longitudinal axis, the proximal end coupled to an end of a catheter;
a plurality of extensions operably coupled to the distal end of the elongated member; and
a sheath having a distal end encircling at least the distal end of the elongated member an in a longitudinally slidable relation therewith, where in a first sliding position, at least the distal end of the elongated member and the plurality of extensions are encircled by the sheath, and in a second sliding position, the plurality of extensions project from the distal end of the sheath,
wherein each of the plurality of extensions extend in a different plane from each of the other extensions in the second sliding position, and
wherein the plurality of extensions are structured such that, in the second sliding position of the sheath when the device is deployed in the pericardial cavity, at least one of the plurality of extensions extend radially outwardly to contact a surface below the pericardial cavity and at least one of the plurality of extensions extends radially outwardly to contact a surface above the pericardial cavity.

2. The pericardial transection device of claim 1, wherein the at least one of the elongated member or one or more of the plurality of extensions are flexibly rigid.

3. The pericardial transection device of claim 1, wherein at least one of the one or more of the plurality of extensions or the elongated member comprises a shape memory material.

4. The pericardial transection device of claim 1, wherein each of the plurality of extensions are spatially separated from each other in a plane in the second sliding position.

5. The pericardial transection device of claim 1, wherein the at least one of the plurality of extensions that extends outwardly to contact the surface above the pericardial cavity is structured for cutting pericardial tissue.

6. The pericardial transection device of claim 1, wherein the plurality of extensions each extend radially outwardly to collectively form a X-or Y-or T-shape in the second sliding position.

7. The pericardial transection device of claim 1, wherein at least one of the plurality of extensions comprises an incision member and at least two of the plurality of extensions are structured to provide any one of: stability, fixation or increase surface contact in the second sliding position.

8. The pericardial transection device of claim 1, wherein at least two of the plurality of extensions extend either obtuse to each other or acute to each other in the second sliding position.

9. The pericardial transection device of claim 1, wherein at least two of the plurality of extensions extend orthogonal to each other in the second sliding position.

10. The pericardial transection device of claim 1, wherein the sheath comprises a radiopaque material that is randomly dispersed or arranged in a pattern.

11. The pericardial transection device of claim 1, wherein at least one of the plurality of extensions comprises an incision member, and wherein said incision member comprises one or more of: a blade, an electrode, and a RF electrode capable of cutting pericardial tissue.

12. The pericardial transection device of claim 1, wherein the device is sterilized.

13. The pericardial transection device of claim 2, wherein at least one of the plurality of extensions comprises an incision member coupled to a controller at the proximal end of the elongated member.

14. A pericardial transection device for use in a pericardial cavity of a heart, said device comprising:
an elongated member having a distal end and a proximal end and extending along a longitudinal axis, the proximal end coupled to an end of a catheter;
a plurality of extensions operably coupled to the distal end of the elongated member; and
a sheath having a distal end encircling at least the distal end of the elongated member an in a longitudinally slidable relation therewith, where in a first sliding position, at least the distal end of the elongated member and the plurality of extensions are encircled by the sheath, and in a second sliding position, the plurality of extensions project from the distal end of the sheath,
wherein the plurality of extensions are structured such that, in the second sliding position when the device is deployed in the pericardial cavity, at least two of the plurality of extensions extend radially outwardly to contact a surface below the pericardial cavity and at least one of the plurality of extensions extends radially outwardly to contact the surface above the pericardial cavity, wherein the at least one of the plurality of extensions structured to contact the surface above the pericardial cavity is structured for cutting pericardial tissue.

15. The pericardial transection device of claim 14, wherein the plurality of extensions each extend radially outwardly to collectively form a X-or Y-or T-shape in the second sliding position.

16. The pericardial transection device of claim 14, wherein at least one of the plurality of extensions comprises an incision member and at least two of the plurality of extensions are structured to provide any one of: stability, fixation or increase surface contact in the second sliding position.

17. The pericardial transection device of claim 14, wherein at least two of the plurality of extensions extend either obtuse to each other or acute to each other in the second sliding position.

18. The pericardial transection device of claim 14, wherein at least one of the plurality of extensions comprises an incision member, and wherein said incision member comprises one or more of: a blade, an electrode, and a RF electrode capable of cutting pericardial tissue.

19. A pericardial transection device for use in a pericardial cavity of a heart, said device comprising:
an elongated member having a distal end and a proximal end and extending along a longitudinal axis, the proximal end coupled to an end of a catheter;
a plurality of extensions operably coupled to the distal end of the elongated member; and
a sheath having a distal end encircling at least the distal end of the elongated member an in a longitudinally slidable relation therewith, where in a first sliding position, at least the distal end of the elongated member and the plurality of extensions are encircled by the sheath, and in a second sliding position, the plurality of extensions project from the distal end of the sheath, and
wherein the plurality of extensions are structured such that, in the second sliding position when the device is deployed in the pericardial cavity, (i) at least two of the plurality of extensions extend radially outwardly to contact a surface below the pericardial cavity and are structured to provide any one of: stability, fixation or increase surface contact in the second sliding position and (ii) at least one of the plurality of extensions extends radially outwardly to contact the surface above the pericardial cavity, wherein the at least one of the plurality of extensions structured to contact the surface above the pericardial cavity is structured for cutting pericardial tissue.

20. The pericardial transection device of claim 19, wherein the plurality of extensions each extend radially outwardly to collectively form a X-or Y-or T-shape in the second sliding position.
